(19) United States
(12) Patent Application Publication
(10) Pub. No.: US 2005/0222632 A1
(43) Pub. Date: Oct. 6, 2005

(54) Device and method for the treatment of cardiac disorders

(76) Inventor: Stan F. Obino, Chanhassen, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 10/515,135

(22) PCT Filed: May 15, 2003
(86) PCT No.: PCT/US03/15341

§ 371 Date: Apr. 25, 2005

(30) Foreign Application Priority Data
May 17, 1992 (US) 60381300

(51) Int. Cl. A61N 1/20

(52) U.S. Cl. 607/28

(57) ABSTRACT

The present invention shows the use of a conductive basket or mesh electrode to actively treat or passively extinguish cardiac arrhythmias.

### CROSS REFERENCE

The present application claims the benefit of, and priority from U.S. Provisional Patent Application 60/381,300 filed May 17, 2002.

### FIELD OF INVENTION

The present invention relates generally to a device and method for using the device to treat or prevent the occurrence of tachy-arrhythmias, including atrial fibrillation and ventricular fibrillation, and to improve cardiac hemodynamics in congestive heart failure.

### BACKGROUND OF THE INVENTION

The heart is an electromechanical pump. In normal conditions, an electrical pulse generated by the sinus node creates an activation front that is conducted in orderly fashion through the atria tissue to the AV node and the His bundle to initiate mechanical pumping. The master pacemaker is located in the atrium (upper chamber). It acts like a spark plug that fires in a regular, rhythmic pattern to regulate the heart's rhythm. This “spark plug” is called the sinoatrial (SA), or sinus node. It sends signals to the rest of the heart so the muscles will contract. Like a pebble dropped into a pool of water, the electrical signal from the sinus node spreads through the atria. If the cardiac tissue was homogenous, the pulsing from the one site should eventually entrain all available tissue. However, this does not happen because cardiac tissue is not homogenous. It is widely recognized that cardiac tissue inhomogeneity is the basic reason for many cardiac disorders. Experimental studies have demonstrated that electrophysiological properties such as conduction velocity, excitability, and refractory period have spatial inhomogeneity. E.g., see M. Wijffels, et al., Circulation, vol. 92, No. 7, October 1995, pp. 1954-1968. This results in areas that may create single, early activation sites, which in turn generate independent wavelets. In general, the arrhythmia propagates because individual wavelets of electrical energy are asynchronously propagated along the walls of the heart that become further fractionated when the wavelet encounters a functional or anatomic obstacle. Fractionated wavelets that diverge into independent paths can be propagated around refractory tissues in a so-called circus movement. This propagation mode requires a critically sized area of excitable (non refractory) tissue to create a re-entrant circuit. Modern techniques for mapping the electrical activity of the heart are now available and in common use as typified by the so-called Constellation catheter by Boston Scientific described in part in U.S. Pat. No. 6,487,441 to Swanson et al.

One of the most common atrial arrhythmias is atrial fibrillation. In the beginning of the 60's Moe suggested the multiple wavelet hypothesis to explain the reentry mechanism of sustained atrial fibrillation. About 25 years later Allessie experimentally confirmed this theory in an isolated Langerdorff perfused canine hearts model. According to this theory, atrial fibrillation can only be sustained when at least 3 different wavelets are wandering in the atrial tissue. Fully developed fibrillation is a state in which many such randomly wandering wavelets coexist. This requires a tissue area large enough to accommodate at least 3 anatomical pathways of adequate length to maintain a reentry mechanism.

This theory confirmed other experimental observations on the role of a critical minimum tissue mass to support atrial fibrillation. After cutting or clamping off a small portion of the wall of fibrillating atrium, Garrey in 1914 demonstrated that fibrillation immediately ceased in the separated portion, while the rest continued to fibrillate. It correlates well with the observation that atrial fibrillation is very difficult to induce and maintain in rabbits, while it is not uncommon in dogs and humans. Traditional therapies have included the use of drugs for increasing the overall refractory periods of tissue to suppress the arrhythmia. In recent years, a surgical technique called the Maze procedure has been used to treat AF by forming lesions in atrial tissue to create smaller areas, each unable to maintain a re-entrant circuit. Also certain multi site pacing techniques have been applied which attempt to interrupt the circus arrhythmia or alternatively attempt to reduce inhomogeneity. See for example U.S. Pat. No. 6,078,837 which uses individually timed stimuli to treat arrhythmias. Other forms of multi-site pacing for arrhythmia prevention are discussed in U.S. Pat. No. 5,584,867, issued to Limousin et at, U.S. Pat. No. 5,683,429 issued to Mehra and U.S. Pat. No. 5,403,356, issued to Hill et al. and in the article “Prevention of Atrial Tachyarrhythmias Related to Advanced Inter-atrial Block by Permanent Atrial Resynchronization”, by Mabo, et al, published in Pace, Vol. 14, April 1991, Part II, p 648. (The Limousin, Mehra and Hill et al. patents are hereby incorporated herein by reference in their entireties.)

When atrial fibrillation cannot be prevented other therapies should be used to break it and to restore normal sinus rhythm. Implantable Cardioverter Defibrillators ICDs are generally capable of delivering the appropriate electrical stimulation/therapy to the patient's heart to terminate the arrhythmias. ICDs consist of an energy storage device, e.g., a capacitor, connected to a shock delivering electrode or electrodes. U.S. Pat. No. 5,545,189 provides a representative background discussion of these and other details of conventional ICDs, and the disclosure of this patent is herein incorporated by reference. The minimum amount of energy required to defibrillate a patient's atrium is known as the atrial defibrillation threshold (ADFT). Although effective, the present electrodes/generator systems require a shock to terminate a given fibrillation episode that is highly painful. U.S. Pat. No. 6,292,691 issued to Pendekanti, et al. describes multiple sites atrial pacing conducted in an independent (asynchronous) manner to maximizing the extent of phase-locked area of atrial tissue. Next, an ADF shock is introduced, if still needed, to achieve atrial defibrillation. This approach should reduce energy requirements for ADFTs.

Multiple site pacing has been also proposed to reduce the incidence of tachyarrhythmias in the ventricle. For example, in U.S. Pat. No. 3,937,226, issued to Funke, multiple electrodes are provided for location around the ventricles. U.S. Pat. No. 4,354,497, issued to Kahn adds sensing electrodes adjacent the septum of the heart and delivers pacing pulses to multiple electrodes spaced around the ventricles in response to sensed depolarizations at the ventricular electrodes which are not preceded by depolarizations sensed at the septum electrodes. An alternative approach to reduce the atrial defibrillation threshold has been described by Zheng X, et al, in Circulation 2001 Aug. 28; 104(9): 1066-70 in the article “Right atrial septal electrode for reducing the atrial defibrillation threshold.

Multi-site pacing in the ventricles has also been proposed to improve hemodynamic function, as in U.S. Pat. No. 4,928,688, issued to Mower, and in the article “Developing Clinical Indication for Multisite Pacing”, by Kappenberger L, et al, published in J Interv Card Electrophysiol 2000 January; 4 Suppl 1:87-93. (The Funke, Rockland and Mower patents are all hereby incorporated herein by reference in their entireties.)

### SUMMARY OF THE INVENTION

It is an object of the invention to produce a device to prevent atrial fibrillation, treat atrial and ventricular tachyarrhythmias, and to improve hemodynamic function in congestive heart failure patients. It is another object of the invention to produce a device that can be either inserted similarly to an intravenous pacemaker lead or percutaneously onto the exterior surface of the heart in the manner of an epicardial pacemaker lead. It is a further object of the invention to allow the use of different embodiments of the same basic device, with a rhythm management device such as a pacemaker in an “active” mode or without a pulse generator or other electrical source of energy in a “passive” mode.

In the first embodiment of the present invention a flexible, radially expandable, conductive mesh or basket is formed which may be applied endocardially to the right atrial chamber and which provide multiple conductivity paths to equalize and reset the cardiac tissue inhomogeneity. In general, the mesh or basket represents a continuous single distal electrode surface mechanically attached to an implantable or temporary catheter introduced transvenously. A proximal ring electrode is located on the terminal site of the catheter close to the mesh or basket. The catheter body is manufactured with the same technology used for standard temporary or permanently implantable pacing leads. The conductive mesh or basket and the proximal ring are individually wired inside the catheter body, and connected with a standard unipolar or bipolar pacing connector. The lead is inserted like a regular pacing lead through a sheath introducer, and the basket is then radially expanded into the atrial cavity in full contact with the endocardial tissue. The size of the mesh, or the distance between basket ribs, will be such that the tissue area inscribed into and surrounded by the conductive wire is too small to host a full re-entrant pathway or circuit. The device is connected to a unipolar or bipolar programmable pacemaker, used preferably in AAT mode. Anytime a regular or premature atrial beat is detected a pacing pulse is applied to the entire mesh or basket, thus equalizing and resetting tissue conductivity. It is also expected that other pacing modality can be used, including burst, or that sub-threshold pacing level energy (micro joules) may be delivered to the network in synchrony with detected atrial beats to ensure termination and prevention of atrial arrhythmias.

The lead can be also connected to an atrial defibrillator to deliver low energy ADF shock. The ADFT will be substantially reduced by the special electrical spatial distribution allowed by the mesh or basket, thus allowing effective painless interruption of atrial fibrillation.

The mesh or basket can be deployed in the right ventricle using the same introduction technique. In this position the device can be connected to a standard pacemaker to manage patients with congestive heart failure. A second similar device can be inserted in the right atrium for atrio-ventricular sensing/pacing for the same application. The rationale is that the special pacing characteristics of this device guarantees a more efficient atrial and/or ventricular systole, and the actual pacing of the left side of the heart through the part of the mesh or basket in contact with the septum. The delivery of energy to the septum recruits the tissues in the opposing chamber.

It is also proposed to use the same construction in the right ventricles to defibrillate ventricular fibrillation. In this application the lead is connected with a ventricular ICD, and/or with any combination of antitachycardia pacemaker.

A second embodiment of the invention includes the same mesh or basket not mechanically attached to the catheter body. Once advanced into the same hollow sheath type introducer, the device is released in the atrial or ventricular cavity, where is left to fully expand toward the endocardial tissue. This device is used as an electrical reference for the tissue in order to remove inhomogeneities and/or transmit a pacing or natural beat wandering in or through a single point in the cavity. This device is intended to prevent atrial arrhythmias, including atrial fibrillation and flutter, when used in the right and/or the left atrium. The same device is used in the left atrium and/or left ventricle when a mesh or basket described as the first embodiment is used in the right site of the heart (atrium and/or ventricle) for the same purposes discussed with former embodiment. It is proposed that such combination increases the efficacy of the pacing or defibrillating therapies by conveying the electrical energy more efficiently on the left heart.

A third embodiment of the invention includes smaller, stent like conductive nets of various shapes to be used in specific cardiac districts (as in the outflow pulmonary tract) to prevent atrial flutter/fibrillation induction. These applications will be of the “passive” type.

A fourth embodiment of the invention envisions an umbrella like device made with the same technology and material of the mesh or basket, where the tip of the umbrella like deployed shape is a screw-in electrode. In endocardial versions of the device the screw points “outward” to be actively fixed on a specific endocardial surface. When used outside the heart the screw points “inward” and the device may be used on an epicardial area. Both of these embodiments are preferably of the “active” type.

### BRIEF DESCRIPTION OF DRAWINGS

Throughout the several views of the drawings like reference numerals refer to identical structure, wherein:

FIG. 1 is a schematic view illustrating both active and passive embodiments of the invention and both basket and mesh features of the device;

FIG. 2 is a schematic view of the heart with an active configuration for pacing or defibrillation;

FIG. 3 is a schematic view of the heart with an active configuration for pacing or defibrillation;

FIG. 4 is a schematic view of the umbrella like fourth embodiment device;

FIG. 5 is a schematic view of the stent-like third embodiment device; and,

FIG. 6 is a schematic view of the heart with two passive devices deployed.

### DETAILED DESCRIPTION OF DRAWINGS

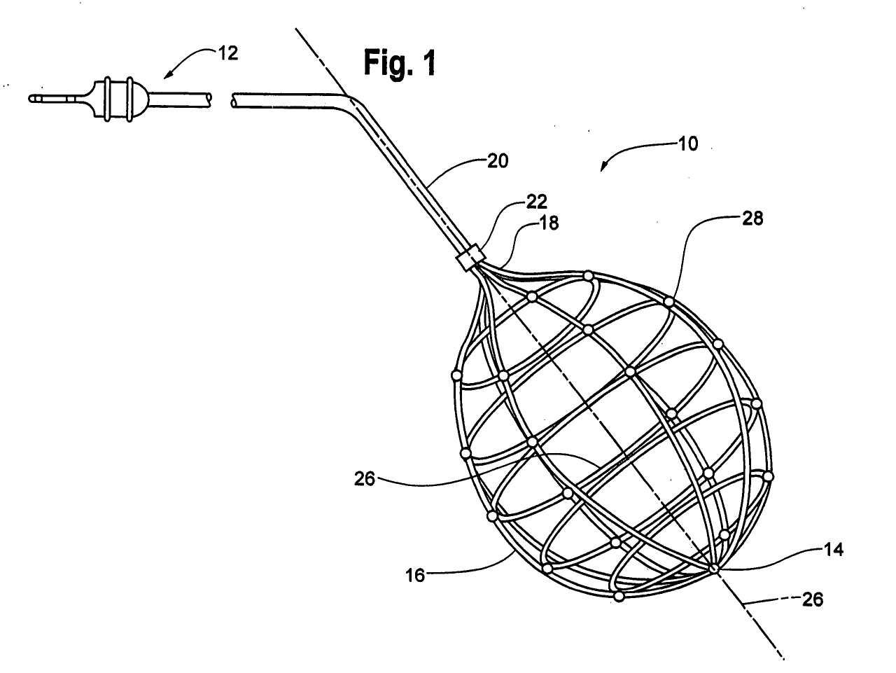

FIG. 1 is a schematic view of the basket device. It is intended to show both “active” and “passive” embodiments as well as both “basket” and “mesh” embodiments. Several features of the device shown in FIG. 1 are optional as explained below.

In this figure the medical device 10 is fully deployed and the device assumes a volume filling shape. The device 10 as depicted has a proximal end terminating in a pacing connector 12. The device also has a distal tip 14 where the individual loops of wire that make up the structure come together. In the figure the structure uses eight loops typified by loop 16 to make up the volume filling structure. However, both greater and lesser numbers of wires may be used. The wire loops meet at the proximal end of the basket 18 where they may connect to the catheter body 20. A ring electrode may optionally be placed on the catheter body as illustrated by ring 22.

Optional circumferential wires may be added to the device as typified by encircling wire ring 26. The individual loop wires exemplified by wire 16 are approximately orthogonal to the wire rings and longitudinal to the main axis 24 of the device. Embodiments of the device where the circumferential rings are present are called “mesh” devices while the embodiment where only the wire loops are present are referred to as “basket” devices. Depending on the particular chamber of implantation, and depending on whether the therapy is “active” or “passive” the basket or mesh device may be preferred.

In general the loops and optional rings, will be all made from resilient material like for example stainless steel, platinum, Nitinol or plastic coated with an electrically conductive material. The overall objective is to use the entire wire surface as a single electrode touching the chamber wall at every point. The whole chamber surface will be then separated in several smaller areas, each bordered and defined by an electric barrier, and individually too small to maintain a re-entrant arrhythmic circuit path. Alternatively, reduced contact surface may be required to improve pacing/sensing characteristics. For this reason total electrode area may need to be limited by incorporating insulating sleeves that may be placed over the wires during construction to define separate electrode nodes such as node 28.

This figure is also intended to depict an embodiment where the basket is detachable for the catheter body 20. The detachment mechanism itself is not illustrated. In this detachable embodiment the basket is deployed and released in the cardiac chamber and it is left in a passive free standing mode of operation. After deployment the catheter 20 is removed from the chamber and the mesh or basket is left behind.

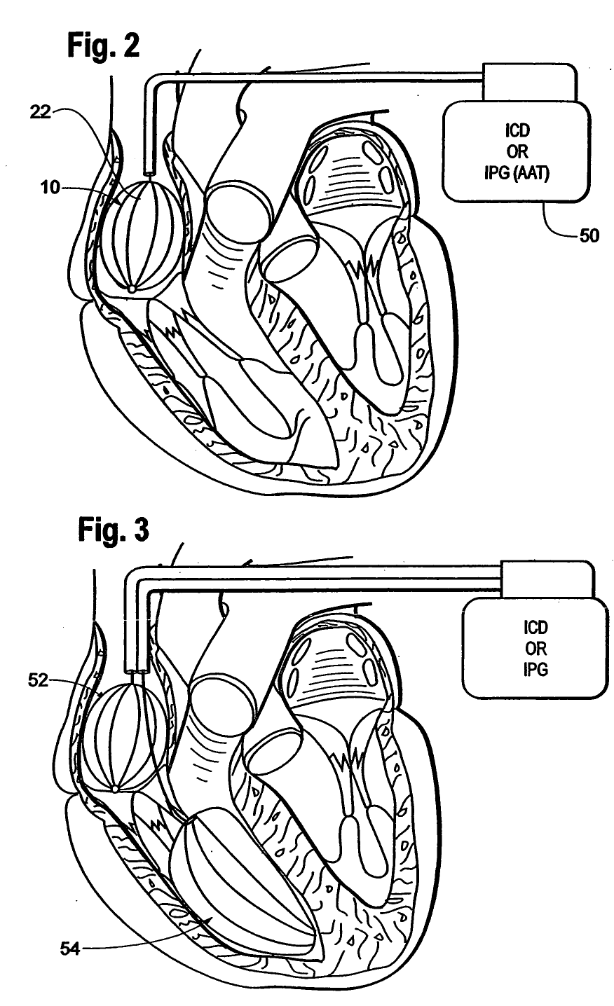

FIG. 2 shows an “active” configuration of the “basket” network device 10 lying in one chamber of the atria (RA) and coupled to a rhythm control device 50 such as a pacemaker or implantable pulse generator (IPG) or implantable Cardioverter defibrillator (ICD). In this preferred embodiment the device 10 is of the “basket” configuration and includes the optional ring electrode to act as an indifferent electrode for AAT pacing or defibrillation. In this configuration the total electrode area of all electrode sites or wires may approximate the area of conventional pacing lead cathode. Greater and lesser areas are contemplated.

FIG. 3 shows a dual chamber active configuration that can be used to provide conventional dual chamber modalities of therapy including DDD, DVI, VDD and VAT modes. In this figure both the atrial and ventricular “baskets” are implanted in the right heart. The atrial basket 52 has a ring electrode while the ventricular basket 54 is operated in the unipolar mode for pacing level energies, but any other combination of unipolar and bipolar modality can be used. It is important to note that the ventricular and atrial baskets have contact with the septum of the heart. It is believed that delivery of pacing energy to the septum will resynchronize the atrial and ventricular chambers.

Also depicted in the figure is the availability of an implantable cardioverter defibrillator ICD that provides higher than pacing energy stimuli. The distribution of the stimuli over the entire basket electrode area should reduce defibrillation thresholds that is a desirable feature.

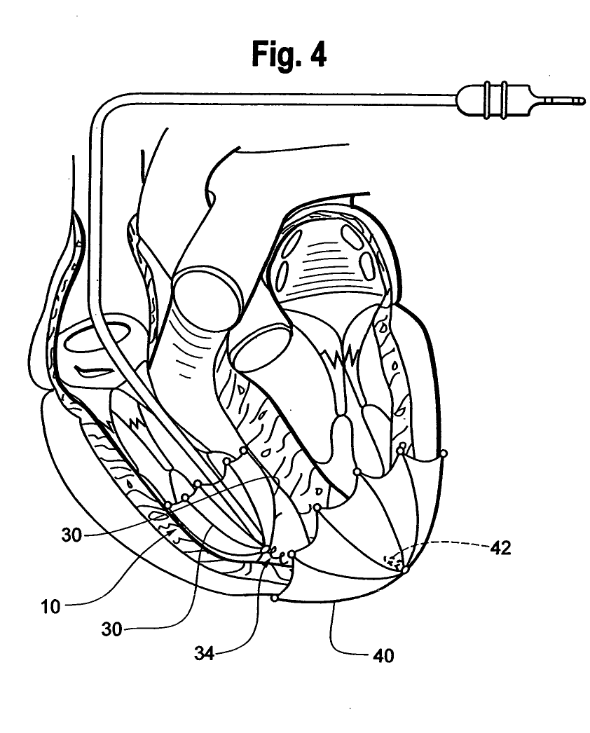

FIG. 4 depicts in the right ventricle a modified basket device with a hemisphere of the device removed to leave an umbrella shaped device. In this configuration the wire loops 30 are cut in half and anchored only at the distal tip 14. An active fixation screw 34 is formed on the distal tip 14 to allow the device 10 to be anchored in the ventricular tissue. Once again a catheter body may be supplied to couple the device 10 to the remote rhythm management device. The same figure shows the epicardial version of the umbrella shape device anchored to the left ventricle epicardium, with an inward screw-in.

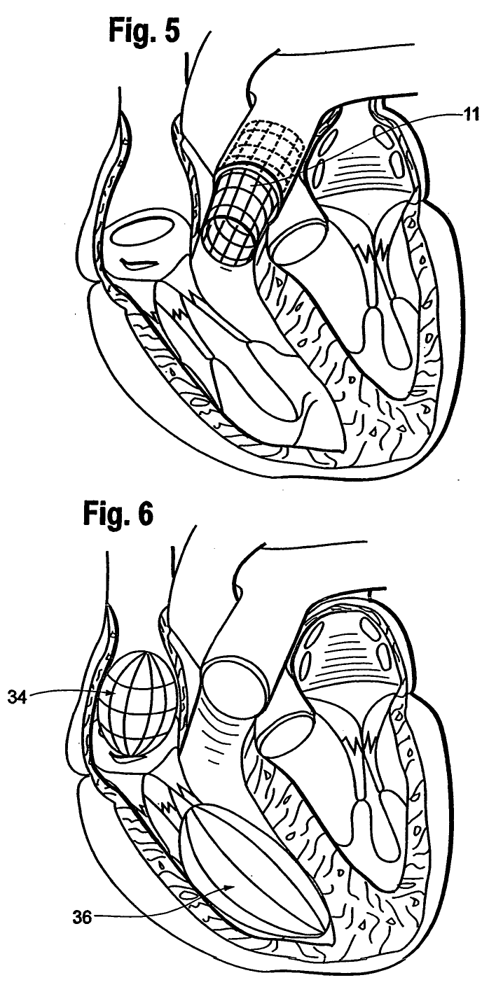

FIG. 5 shows a single volume-enclosing stent like device 11 that includes an annular electrically conductive mesh expanding inside a vessel like the pulmonary outflow tract. It is widely recognized that certain anatomical structures around the heart are the origin of potentially arrhythmogenic early activation sites. Isolating these districts through the use of a passive iso-electric network will prevent the creation and maintenance of arrhythmias.

FIG. 6 shows two passive networks located in the right heart. The atrial device 34 is of the passive mesh type while the ventricular device 36 is a basket configuration. The basket and mesh interact with the conduction through the heart tissues to prevent the formation of arrhythmia. It should be understood that the devices may be adapted for application outside the heart where the same benefits will obtain.

Although the devices are intended to be used in the right heart in the preferred modes of operation in certain applications the devices may be introduced into the left heart. It should also be apparent that departures from the construction depicted are within the scope of the claims. It should also be apparent that other conventional lead systems may be used in conjunction with the invention included coronary sinus leads to assist in ventricular resynchronization.

1-15. (canceled)

16. A passive implantable medical device for insertion into a chamber of a patient's heart comprising:
a radially expandable basket formed from a set of resilient wire loop elements coupled together at a distal tip, and coupled together at a proximal tip, said basket having a substantially rounded shape when deployed to conform to the shape of a heart chamber;
each wire element being mechanically and electrically coupled to all other wire elements at the distal and/or proximal tip, thereby forming an approximately balloon shaped electrically conductive network positioned in contact with an atrial or ventricular surface upon insertion and deployment, whereby low voltage low current energy arising in the tissues or impressed on the tissues are distributed over the tissues in contact with the conductive network.

17. A passive implantable medical device for insertion into a chamber of a patient's heart comprising:
a radially expandable basket formed from a set of resilient wire loop elements coupled together at a distal tip, and coupled together at a proximal tip, said basket having a substantially rounded shape when deployed to conform to the shape of a heart chamber;
each wire element being mechanically and electrically coupled to all other wire elements at the distal and/or proximal tip, thereby forming an approximately balloon shaped electrically conductive network positioned in contact with an atrial or ventricular surface upon insertion and deployment;
a plurality of annular wire rings positioned at orthogonal angles to said resilient wire loop elements, thereby forming a mesh network thereby connecting all of the elements of the network forming a set of network cells and bringing them to the same potential;
wherein the heart tissue surrounded and inscribed in each network cell is lower than the critical value necessary to maintain a single wavelet of a reentrant arrhythmia.

18. The device of claim 16 further comprising;
a plurality of insulative sleeves paced intermittently along each of said wire loop elements to segment each wire element into a set of electrically interconnected electrode elements;
a catheter body having a distal end coupled to said basket at said proximal tip of said basket or mesh for electrically coupling said set of resilient wire elements to a remote rhythm management device;
said catheter body having a proximal end terminated in a pacing connector;
said basket or mesh, thereby functioning as a distal electrode for said rhythm management device is located in the right atrial chamber of the heart;
a rhythm management device coupled to said pacing connector for delivering a temporary or permanent AAT pacing therapy to the chamber containing said active basket or mesh, thereby preventing the onset of arrhythmia.

19. The device of 18 further comprising;
a proximal ring electrode located on the body of the catheter proximate said catheter body distal end, said ring electrode being electrically independent of said mesh and separately terminated in said pacing connector.

20. The device of claim 18 further comprising:
said passive basket or mesh located in the left atrial heart chamber for supplying a passive therapy in conjunction with the active therapy delivered by said rhythm management device in the right atrial chamber.

21. The device of claim 18 wherein said rhythm management device delivers a polarization voltage or a therapy selected from the group of:
sub-threshold stimulation pulses, super-threshold stimulation pulses, burst antitachycardia stimulation.

22. The device of claim 18 wherein said basket or mesh is located in the right ventricle of the heart and the pacing modality of the rhythm management device is changed to VVI, DDD or equivalent for the treatment of symptoms associated with congestive heart failure.

23. The device of claim 22 further comprising:
a passive basket or mesh located in the left ventricle for supplying a passive therapy in conjunction with the active therapy in the right ventricular chamber.

24. The device of claim 16 wherein at least one device is placed and deployed in a chamber selected from the group consisting of: the RV, the LV, the RA, the LA.

25. The device of claim 16 wherein at least two of said devices are placed and deployed in chambers selected from the group consisting of: the RV, the LV, the RA, the LA; such that current sourced in one chamber is returned to supply by the other of said devices.

26. The device of claim 16 wherein at least three devices are placed and deployed in chambers selected from the group consisting of: the RV, the LV, the RA, the LA.

27. The device of claim 16 wherein one of said devices is placed and deployed in each chamber of the heart namely: the RV, the LV, the RA, the LA.

28. A medical device for insertion and implantation into a vessel in the pulmonary outflow tract comprising:
a metallic mesh of resilient wires having a stent-like shape providing electrical and mechanical contact with cardiac endocardial surfaces, thereby preventing the initiation of atrial or ventricular arrhythmias.

29. An implantable medical device for application into a chamber of a patient's heart comprising:
a radially expandable basket formed from a set of resilient wire elements coupled together at a distal tip, said basket having a substantially hemispheric shape when deployed to conform to the shape of a heart chamber;
each wire element being mechanically and electrical coupled to all other wire elements at the distal tip, thereby forming an approximately hemispheric surface shaped electrically conductive mesh or mesh positioned in contact with an interior or exterior surface of the heart;
a screw anchor coupled to said distal tip for retaining said mesh in contact with cardiac tissues.

30. The device of claim 29 wherein said device is positioned in contact with an epicardial surface upon deployment.

31. The device of claim 29 wherein said device is positioned in contact with an endocardial surface upon deployment.

32. The device of claim 17 further comprising;
a plurality of insulative sleeves paced intermittently along each of said wire loop elements to segment each wire element into a set of electrically interconnected electrode elements;
a catheter body having a distal end coupled to said basket at said proximal tip of said basket or mesh for electrically coupling said set of resilient wire elements to a remote rhythm management device;
said catheter body having a proximal end terminated in a pacing connector;
said basket or mesh, thereby functioning as a distal electrode for said rhythm management device is located in the right atrial chamber of the heart;
a rhythm management device coupled to said pacing connector for delivering a temporary or permanent AAT pacing therapy to the chamber containing said active basket or mesh, thereby preventing the onset of arrhythmia.

33. The device of 32 further comprising;
a proximal ring electrode located on the body of the catheter proximate said catheter body distal end, said ring electrode being electrically independent of said mesh and separately terminated in said pacing connector.

34. The device of claim 32 further comprising:
said passive basket or mesh located in the left atrial heart chamber for supplying a passive therapy in conjunction with the active therapy delivered by said rhythm management device in the right atrial chamber.

35. The device of claim 18 further comprising:
said passive basket or mesh located in the left atrial heart chamber for supplying a passive therapy in conjunction with the active therapy delivered by said rhythm management device in the right atrial chamber.

36. The device of claim 33 further comprising:
said passive basket or mesh located in the left atrial heart chamber for supplying a passive therapy in conjunction with the active therapy delivered by said rhythm management device in the right atrial chamber.

37. The device of claim 32 wherein said rhythm management device delivers a polarization voltage or a therapy selected from the group of:
sub-threshold stimulation pulses, super-threshold stimulation pulses, burst antitachycardia stimulation.

38. The device of claim 33 wherein said rhythm management device delivers a polarization voltage or a therapy selected from the group of:
sub-threshold stimulation pulses, super-threshold stimulation pulses, burst antitachycardia stimulation.

39. The device of claim 32 wherein said rhythm management device delivers a polarization voltage or a therapy selected from the group of:
sub-threshold stimulation pulses, super-threshold stimulation pulses, burst antitachycardia stimulation.

40. The device of claim 20 wherein said rhythm management device delivers a polarization voltage or a therapy selected from the group of:
sub-threshold stimulation pulses, super-threshold stimulation pulses, burst antitachycardia stimulation.

41. The device of claim 32 wherein said rhythm management device delivers a polarization voltage or a therapy selected from the group of:
sub-threshold stimulation pulses, super-threshold stimulation pulses, burst antitachycardia stimulation.

42. The device of claim 35 wherein said rhythm management device delivers a polarization voltage or a therapy selected from the group of:
sub-threshold stimulation pulses, super-threshold stimulation pulses, burst antitachycardia stimulation.

43. The device of claim 36 wherein said rhythm management device delivers a polarization voltage or a therapy selected from the group of:
sub-threshold stimulation pulses, super-threshold stimulation pulses, burst antitachycardia stimulation.

44. The device of claim 32 wherein said basket or mesh is located in the right ventricle of the heart and the pacing modality of the rhythm management device is changed to VVI, DDD or equivalent for the treatment of symptoms associated with congestive heart failure.

45. The device of claim 19 wherein said basket or mesh is located in the right ventricle of the heart and the pacing modality of the rhythm management device is changed to VVI, DDD or equivalent for the treatment of symptoms associated with congestive heart failure.

46. The device of claim 33 wherein said basket or mesh is located in the right ventricle of the heart and the pacing modality of the rhythm management device is changed to VVI, DDD or equivalent for the treatment of symptoms associated with congestive heart failure.

47. The device of claim 44 further comprising:
a passive basket or mesh located in the left ventricle for supplying a passive therapy in conjunction with the active therapy in the right ventricular chamber.

48. The device of claim 25 further comprising:
a passive basket or mesh located in the left ventricle for supplying a passive therapy in conjunction with the active therapy in the right ventricular chamber.

49. The device of claim 46 further comprising:
a passive basket or mesh located in the left ventricle for supplying a passive therapy in conjunction with the active therapy in the right ventricular chamber.

50. The device of claim 17 wherein at least one device is placed and deployed in a chamber selected from the group consisting of: the RV, the LV, the RA, the LA.

51. The device of claim 17 wherein at least two of said devices are placed and deployed in chambers selected from the group consisting of: the RV, the LV, the RA, the LA; such that current sourced in one chamber is returned to supply by the other of said devices.

52. The device of claim 17 wherein at least three devices are placed and deployed in chambers selected from the group consisting of: the RV, the LV, the RA, the LA.

53. The device of claim 17 wherein one of said devices is placed and deployed in each chamber of the heart namely: the RV, the LV, the RA, the LA.
